(19) United States
(12) Patent Application Publication
(10) Pub. No.: US 2024/0050273 A1
(43) Pub. Date: Feb. 15, 2024

(54) OPHTHALMIC SURGICAL PROBE

(71) Applicant: Alcon Inc., Fribourg (CH)

(72) Inventors: Paul R. Hallen, Colleyville, TX (US); Firas Rahhal, Tarzana, CA (US); Steven T. Charles, Memphis, TN (US)

(21) Appl. No.: 18/357,435

(22) Filed: Jul. 24, 2023

Related U.S. Application Data
(60) Provisional application No. 63/370,827, filed on Aug. 9, 2022.

(51) Int. Cl. A61F 9/007

(52) U.S. Cl. CPC A61F 9/00736

(57) ABSTRACT

Embodiments of the present disclosure generally relate to an ophthalmic surgical probe for extrusion. In certain embodiments, the probe includes a beveled probe tip that increases the area of vacuum generation at a target site without increasing probe gauge, thus facilitating improved tissue engagement. In certain embodiments, the probe further includes one or more texturized surfaces for improved “grabbing” and manipulation of target tissues, and/or one or more surfaces formed of polymeric materials to reduce unwanted damage and enhance the safety of use thereof.

### BACKGROUND

The vitreous body, often referred to as the vitreous humor or simply “the vitreous,” is a transparent, colorless, and gelatinous mass that fills the space between the lens and the retina of the eyeball. The vitreous makes up about 80% of the volume of the eyeball and helps maintain the round shape of the eye. Additionally, the vitreous assists in absorbing external mechanical shocks to the eye, provides nutrients to the lens, and supports the retina.

The vitreous is mostly comprised of water with trace amounts of collagen and hyaluronic acid, which provide the vitreous with its gelatinous structure. Over time, however, the vitreous liquefies and condenses (e.g., shrinks) due to age and normal wear and tear. Eventually, the vitreous cannot fill the volume of the eye's vitreous cavity, and so the vitreous separates from the retina, also known as “posterior vitreous detachment” or “PVD.” PVD is common for older adults, and can lead to more serious complications, such as retinal detachment, where the retina peels away from underlying layers of supporting tissues.

When treatment of PVD is necessary, ophthalmic surgeons typically utilize a vitrectomy probe to completely separate the detaching vitreous from the retina, cut the separated vitreous into smaller fragments, and then suction the fragmented vitreous out of the eye. To separate the vitreous from the retina, the cutter of the vitrectomy probe is deactivated, and the port of the probe is brought in close proximity to the detaching vitreous to “grab” the vitreous and peel it away. However, due to the design of traditional vitrectomy probes, and more particularly, the relatively small port sizes thereof, which diminish greatly with smaller probe gauges, it is extremely difficult to grab and peel the vitreous from the retina effectively without causing damage to the retina.

Accordingly, there is a need in the art for improved ophthalmic devices to manipulate and extrude the vitreous and other ocular materials/tissues during surgical procedures.

### SUMMARY

The present disclosure relates to microsurgical tools, and more specifically, to ophthalmic microsurgical devices and methods of use thereof.

In certain embodiments, a vitrectomy probe for manipulating ocular tissues is provided. The vitrectomy probe includes a handpiece configured to be held by a user and a tube defining a longitudinal axis. The tube includes a proximal end coupled to the handpiece, and a distal end opposite the proximal end and comprising a distal tip, wherein the distal tip is beveled and comprises an end face at least partially defining a port. The port has an elongated shape.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A illustrates a conventional surgical probe during an ophthalmic surgical procedure to treat posterior vitreous detachments.

FIG. 1B illustrates an enlarged side view of the surgical probe in FIG. 1A.

FIG. 2A illustrates a side view of an exemplary surgical probe, in accordance with certain embodiments of the present disclosure.

FIG. 2B illustrates a side view of another exemplary surgical probe, in accordance with certain embodiments of the present disclosure.

FIGS. 3A-3C illustrate enlarged cross-sectional side views of exemplary configurations of probe tips for the probes in FIGS. 2A and 2B, in accordance with certain embodiments of the present disclosure.

FIG. 4 illustrates an enlarged cross-sectional side view of an exemplary configuration for a probe tip of the probes in FIGS. 2A and 2B, in accordance with certain embodiments of the present disclosure.

FIGS. 5A-5F illustrate enlarged perspective views of exemplary configurations of probe tips for the probes in FIGS. 2A and 2B, in accordance with certain embodiments of the present disclosure.

FIG. 6A illustrates a magnified perspective view of an exemplary textured surface for the probes in FIGS. 2A and 2B, in accordance with certain embodiments of the present disclosure.

FIG. 6B illustrates enlarged cross-sectional side view of the exemplary textured surface in FIG. 6A, in accordance with certain embodiments of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Note that, as described herein, a distal end, segment, or portion of a component refers to the end, segment, or portion that is closer to a patient's target tissue during use thereof. On the other hand, a proximal end, segment, or portion of the component refers to the end, segment, or portion that is distanced further away from the patient's target tissue.

As used herein, the term “about” may refer to a +/−10% variation from the nominal value. It is to be understood that such a variation can be included in any value provided herein.

The present disclosure relates to microsurgical tools, and more specifically, to ophthalmic microsurgical probes for manipulation of ocular materials/tissues and methods of use thereof.

As described above, during certain procedures, ophthalmic surgeons may utilize vitrectomy probes to separate the vitreous from the retina (e.g., create a posterior vitreous detachment, or “PVD”), prior to cutting and suctioning the vitreous from the eye. While effective for cutting operations, vitrectomy probes may be ineffective for engaging and extruding tissues and other ocular materials, such as the vitreous, and may even facilitate unwanted damage to tissues (e.g., the retina) adjacent to an operation site. The devices described herein address the deficiencies of certain existing methods and designs described above, and further reduce the risk of unwanted damage to peripheral tissues, by providing probes designed for effective engagement of the vitreous and other materials. Such probes include beveled probe tips that increase the area of vacuum generation at a target site without increasing probe gauge, thus facilitating improved tissue engagement as compared to other devices of similar gauge. In certain embodiments, the probes described herein further include one or more texturized surfaces for improved “grabbing” and manipulation of target tissues, and/or one or more surfaces formed of polymeric materials to reduce unwanted damage and enhance the safety thereof.

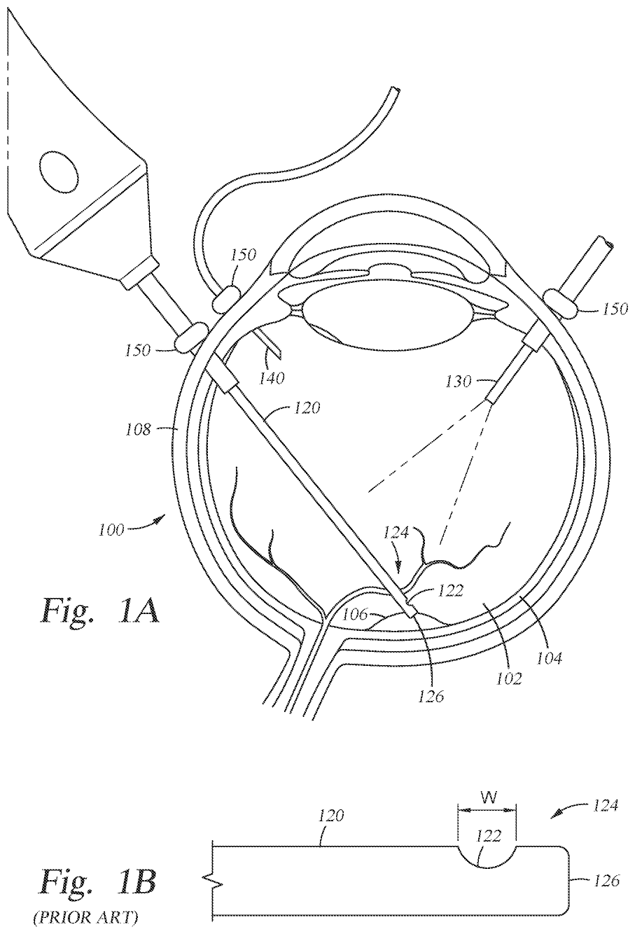

FIG. 1A illustrates a cross-sectional side view of exemplary eye 100 undergoing an ophthalmic procedure in which vitreous 102 is separated from retina 104 by conventional methods to form posterior vitreous detachment (PVD) 106 prior to cutting and suctioning vitreous 102 from eye 100. As shown in FIG. 1A, various microsurgical instruments are inserted into eye 100, including vitrectomy probe 120 for cutting and removing vitreous 102, endoilluminator 130 for providing illumination inside eye 100, and infusion cannula 140 for replacing fluid within eye 100 with saline solution and for maintaining intraocular pressure. Vitrectomy probe 120, endoilluminator 130, and infusion cannula 140 are typically inserted into eye 100 through respective trocar cannulas 150 that are inserted into incisions in sclera 108, as would be understood by skilled persons.

To separate vitreous 102 from retina 104 and create PVD 106, a surgeon may deactivate the cutter of vitrectomy probe 120 and bring port 122 at distal end 124 of the probe near the desired portion of vitreous 102 to “grab” the vitreous. Thereafter, vitrectomy probe 120 may be carefully pulled away from retina 104 to peel vitreous 102 from retina 104. However, due to port 122 being disposed through a sidewall of vitrectomy probe 120 rather than, e.g., distal end face 126, as well as the relatively small size of port 102 as a function of the gauge of vitrectomy probe 120, it may be extremely difficult to extrude vitreous 102 from retina 104 with vitrectomy probe 120 effectively without causing damage to retina 104.

FIG. 1B illustrates an enlarged side view of distal end 124 of vitrectomy probe 120 in FIG. 1A to better depict the arrangement of port 122. As shown, port 122 of vitrectomy probe 120 is disposed through a sidewall of the probe, rather than distal end face 126. Thus, when attempting to manipulate/extrude tissues or other materials within eye 100, e.g., vitreous 102, the surgeon must carefully position, rotate, and angle vitrectomy probe 120 such that port 122 is adjacent to a desired tissue or other material in order to “grab” the tissue or material. Not only that—the surgeon must also consider how to grab the tissue or other material while maintaining visualization of the tissue or material, without obstruction by probe 120. And, as previously mentioned, the relatively small size of port 122, represented as width W in FIG. 1B, may provide suboptimal vacuum generation for engagement with tissues or other materials, thereby further increasing the difficulty in manipulating tissues or other materials with vitrectomy probe 120.

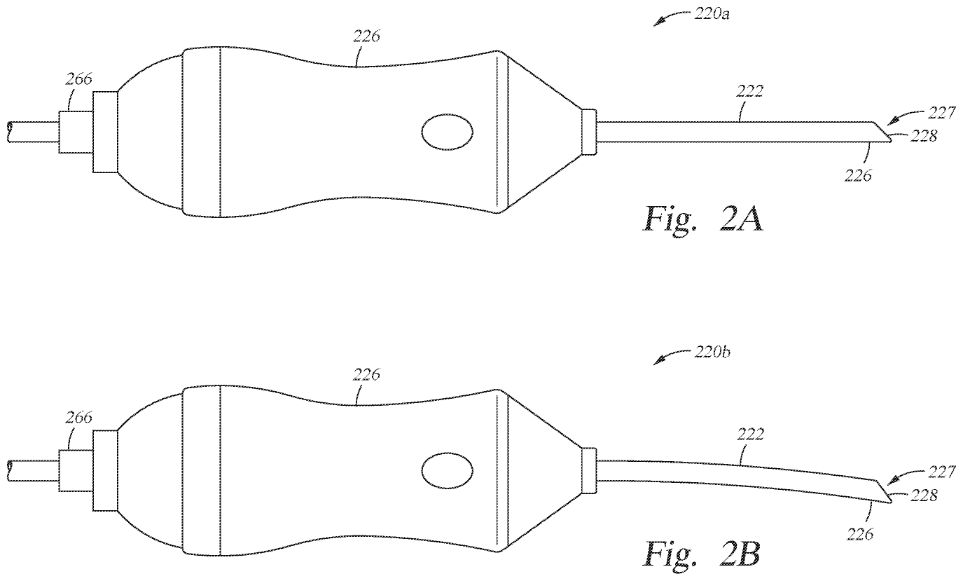

FIG. 2A illustrates a side view of an improved surgical probe 220a, in accordance with certain embodiments of the present disclosure. Probe 220a includes an elongated member that may be inserted into an eye, e.g., through a trocar cannula, for engaging and manipulating the vitreous and other tissues and/or materials. In certain embodiments, probe 220a is configured to create a posterior vitreous detachment, or PVD.

As shown, in certain embodiments, probe 220a comprises a hollow, cylindrical (e.g., non-segmented) tube 222 defining a longitudinal axis of probe 220a and having an outer diameter less than about 20 gauge. For example, in certain embodiments, tube 222 has a diameter of about 23 gauge, 25 gauge, 27 gauge, or less. In certain embodiments, tube 222 is segmented into two or more segments having outer diameters of different sizes. For example, in certain embodiments, a first proximal segment of tube 222 may have an outer diameter of about 23 or 25 gauge, while a second distal segment of tube 222 may have an outer diameter of about 25 or 27 gauge, respectively. In still other embodiments, however, probe 220a comprises a hollow triangular, quadrilateral, or polygonal tube having a plurality of longitudinal facets. Note that, as described herein, a distal segment, portion, or end of a component refers to the segment, portion, or end that is closer to a patient's target tissue during use thereof. On the other hand, a proximal segment, portion, or end of the component refers to the segment, portion, or end that is distanced further away from the patient's target tissue.

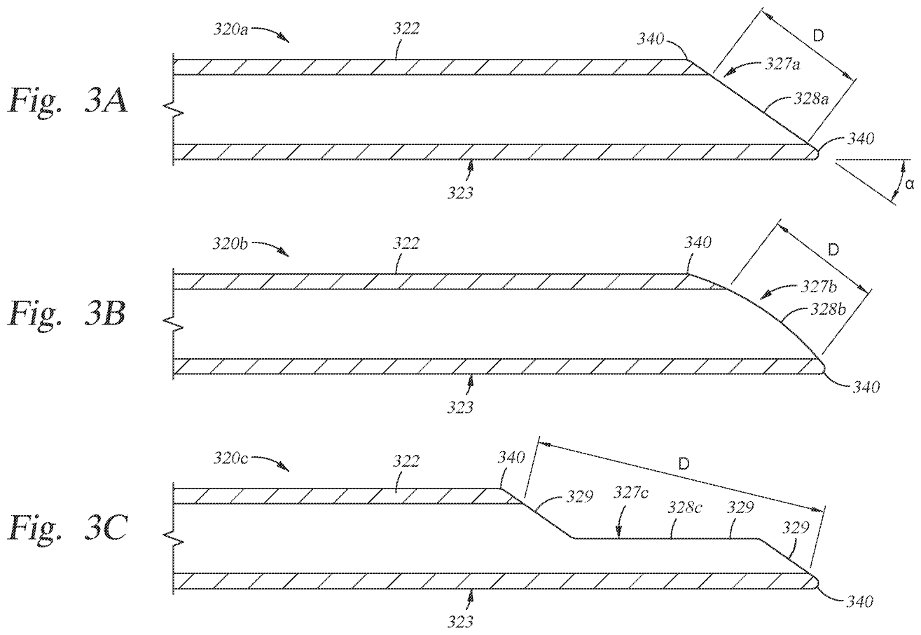

Tube 222 further comprises distal tip 226 at a distal end thereof. Distal tip 226 comprises end face 227 through which port 228 is disposed. Port 228, which is partially defined by end face 227, facilitates the provision of vacuum at a target tissue or material within a patient's eye for “grabbing” and manipulating the tissue or material during ophthalmic procedures. As shown, distal tip 226 is beveled (e.g., angled) at an angle that is non-normal relative to a major (longitudinal) axis 221 of probe 220a, thereby causing port 228 to have an elongated, e.g., ellipsoid shape. The beveled morphology of distal tip 226 and thus, the elongated shape of port 229, increases the surface area of vacuum generation at port 228 without requiring an increase in probe gauge. Accordingly, probe 220a enables improved suction or “purchase” of ocular tissues/materials and thus, easier manipulation thereof, at smaller probe gauges. For clarity, enlarged cross-sectional views of distal tip 226 and port 228 are illustrated in FIGS. 3A-3C, which are described in further detail below.

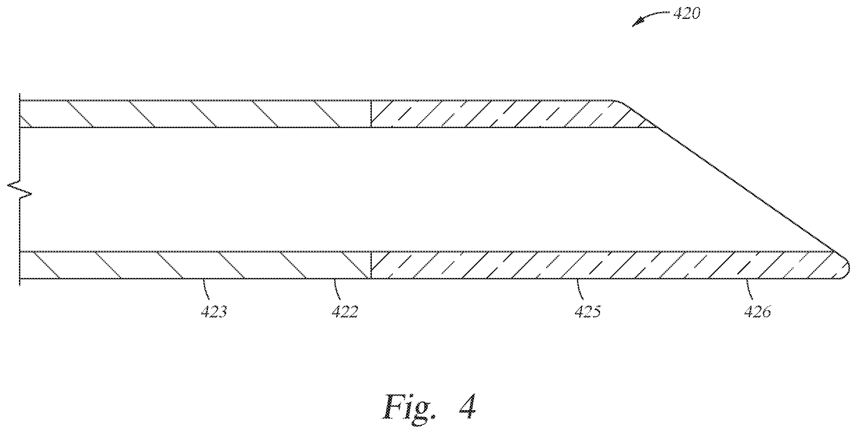

Tube 222 of probe 220a may be formed of any materials suitable for performing ophthalmic procedures. In certain embodiments, tube 222 comprises a plastic or polymeric material. In such embodiments, a portion or substantially all of tube 222 may be translucent or transparent. In certain other embodiments, tube 222 comprises more conventional surgical-grade materials, such as aluminum, stainless steel (e.g., 316 or 316 L stainless steel), or other alloys. In particular examples, tube 222 is formed of Phynox, Elgiloy, or other suitable cobalt-chromium-nickel alloys. In particular examples, tube 222 is formed of nitinol or other suitable nickel-titanium alloys. In further embodiments, the tube 222 may comprise a combination of metallic and polymeric materials, as illustrated and described with reference to FIG. 4.

As further shown in FIG. 2A, a proximal end of tube 222 may, in certain embodiments, be partially and longitudinally disposed through a distal end of handpiece 260, and may be directly or indirectly attached thereto within an interior lumen of handpiece 260. In certain embodiments, handpiece 260 is a hand piece having an outer surface configured to be held by a user, such as a surgeon. For example, handpiece 260 may be ergonomically contoured to substantially fit the hand of the user. In certain embodiments, the outer surface may be textured or have one or more gripping features formed thereon, such as one or more grooves and/or ridges. Handpiece 260 may be made from any materials commonly used for such instruments and suitable for ophthalmic surgery. For example, handpiece 260 may be formed of a lightweight aluminum, a polymer, or other suitable material. In some embodiments, handpiece 260 may be sterilized and used in more than one surgical procedure, or may be a single-use device. Handpiece 260 further includes one or more ports 266 at a proximal end thereof for providing ingress/egress for a vacuum supply lines to be routed into an interior lumen of handpiece 260. For example, port 266 may provide a connection between handpiece 260 (and thus, probe 220a) and a vacuum supply line of a vacuum source within a surgical console.

FIG. 2B illustrates a side view of another exemplary surgical probe 220b, in accordance with certain embodiments of the present disclosure. Surgical probe 220b is substantially similar to surgical probe 220a, but for the presence of curvature 224 in tube 222. Accordingly, tube 222 may be a curved cylindrical, triangular, quadrilateral, or polygonal tube. In certain embodiments, curvature 224 is formed at and/or adjacent to the distal end of tube 222, e.g., within 5-10 mm (millimeters) of the distal tip 226 of tube 222. Generally, curvature 224 may be shaped to match a curvature of the patient's eye, e.g., a retinal surface of eye 100 in FIG. 1A.

FIGS. 3A-3C illustrate enlarged cross-sectional side views of exemplary configurations of probes 320a, 320b, and 320c, which are representative of the surgical probes in FIGS. 2A and 2B, in accordance with certain embodiments of the present disclosure. More particularly, FIGS. 3A-3C depict distal tips 326a, 326b, and 326c of probes 320a, 320b, and 320c, respectively. Each distal tip 326a, 326b, and 326c includes a corresponding end face 327a, 327b, or 327c, respectively, through which a port 328a, 328b, or 328c, respectively, is disposed.

As shown in FIG. 3A, distal tip 326a of probe 320a is beveled such that end face 327a, which is substantially planar in this example, is disposed at a non-normal (non-perpendicular) angle α relative to a major axis 322 of probe 320a. Generally, the beveling of distal tip 326a creates a larger surface area, represented as dimension “D” in FIG. 3A, for port 328a, thus enabling greater vacuum generation thereat, for improved “grabbing” of ocular tissues and other materials during ophthalmic procedures at smaller probe gauges. Additionally, during many ophthalmic procedures requiring manipulation of tissues, the surgical probe is inserted into the eye through an incision and/or cannula disposed in the superotemporal quadrant of the eye, and so the distal tip of the surgical probe is brought toward a target tissue or material (e.g., the back surface of the vitreous) at an angle. Here, by beveling distal tip 326a of probe 320a such that end face 327a is disposed at an angle, probe 320a facilitates greater purchase (e.g., suction) of a target tissue or material, since end face 327a is configured to face the target tissue or material and create a “sealed” vacuum suction thereon. Accordingly, probe 320a facilitates improved manipulation of tissues and other ocular tissues as compared to conventional probes.

In certain embodiments, angle α is between about 0° and about 90° relative to the normal of major axis 322, such as between about 5° and about 70° relative to the normal of major axis 322, such as between about 10° and about 60° relative to the normal of major axis 322, such as between about 20° and about 40° relative to the normal of major axis 322, such as about 30° relative to the normal of major axis 322. In certain embodiments, angle α is between about 10° and about 30° relative to the normal of major axis 322, such as between about 15° and about 25° relative to the normal of major axis 322, such as about 18° relative to the normal of major axis 322.

As described above, end face 327a of probe 320a is substantially planar, and is connected to outer surface 323 of tube 322 by lateral edge 340, which may be rounded. However, other end face profiles/morphologies are also contemplated, such as those shown in FIGS. 3B and 3C. For example, in FIG. 3B, end face 327b comprises a curved or rounded profile. In certain embodiments, end face 327b comprises an outward (e.g., convex) curvature, which may, in certain embodiments, match the curvature of a patient's eye, e.g., a retinal surface of eye 100. In certain other embodiments, end face 327b may comprise an inward (e.g., concave) curvature. In the example of FIG. 3C, end face 327c comprises a staggered, or stepped, profile having a plurality of incremental, or stepped, segments 329. In such embodiments, segments 329 may be substantially planar as shown in FIG. 3C, or segments 329 may be curved or rounded. Note that although three segments 329 are depicted, more or less segments are also contemplated.

FIG. 4 illustrates an enlarged cross-sectional side view of an exemplary configuration of probe 420, which is representative of the surgical probes in FIGS. 2A and 2B, in accordance with certain embodiments of the present disclosure. As shown, probe 420 comprises tube 422 having portions thereof formed of at least two different materials. More particularly, tube 422 comprises a first, proximal portion 423 formed of a first material, and a second, distal portion 425 (which includes distal tip 426) formed of a second material. In certain embodiments, proximal portion 423 is formed of a surgical-grade metallic material, such as aluminum, stainless steel (e.g., 316 or 316 L stainless steel), phynox, or other alloys, while distal portion 425 is formed of a plastic or polymeric material. In such embodiments, proximal portion 423 provides the necessary stiffness for maneuvering probe 420 within the intraocular space during an ophthalmic procedure, while distal portion 425, which may be contacted against various tissues in the eye, provides a degree of pliability to reduce the risk of damage to these tissues. Furthermore, utilization of a polymeric distal portion 425 may facilitate improved engagement with ocular tissues and other materials, as a softer probe tip is more likely to “grab” such tissues and materials. Accordingly, the exemplary prove 420 in FIG. 4 may facilitate improved safety and efficiency during certain ophthalmic procedures as compared to more conventional probes.

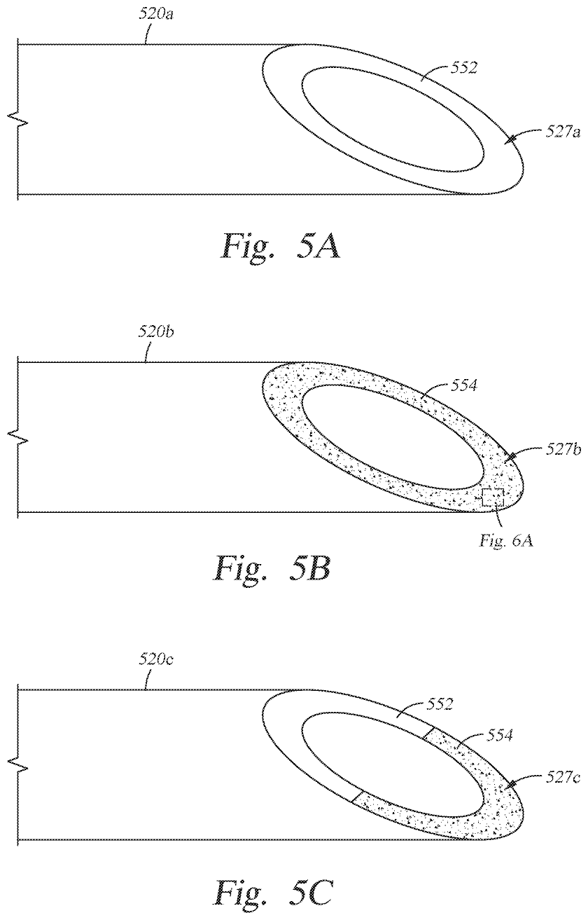

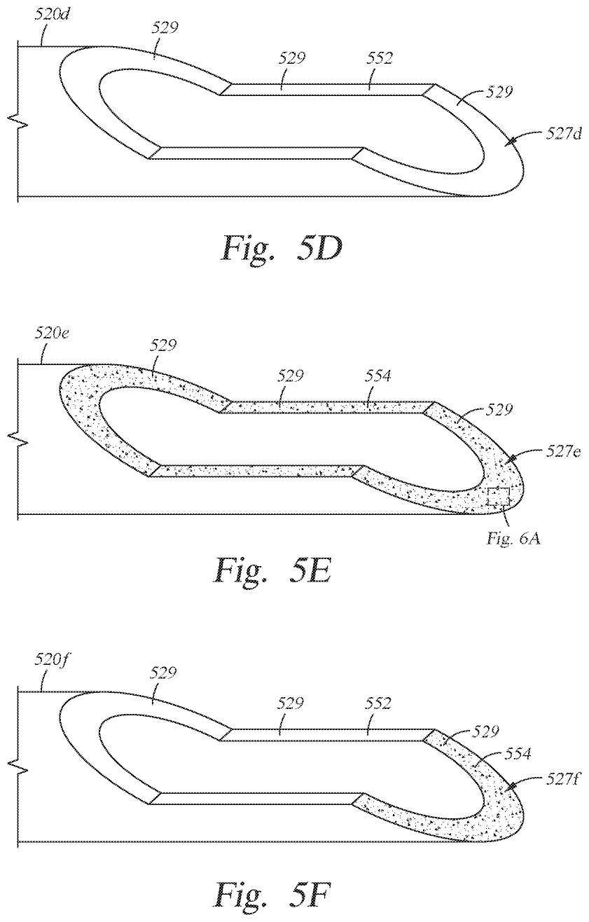

FIGS. 5A-5F illustrate enlarged perspective views of exemplary configurations of probes, which are representative of the surgical probes in FIGS. 2A and 2B, in accordance with certain embodiments of the present disclosure. More particularly, FIGS. 5A-5F illustrate end faces 527a, 527b, 527c, 527d, 527e, and 527f of probes 520a, 520b, 520c, 520d, 520e, and 520f, respectively. End faces 527a-527c are planar in profile, as described with reference to FIG. 3A above, while end faces 527d-527f are staggered, or stepped, as described with reference FIG. 3C above.

As shown, planar end face 527a comprises substantially smooth, or untextured, surface 552 across an entire surface area thereof. Conversely, planar end face 527b comprises textured surface 554 across an entire surface area thereof. Alternatively, planar end face 527b comprises both smooth surface 552 and textured surface 554 across different portions of a surface area thereof. Similarly, staggered end face 527d, which includes a plurality of segments 529, comprises smooth surface 554 across an entire surface area thereof. Meanwhile, staggered end face 527e comprises textured surface 554 across an entire surface area thereof, and staggered end face 527f comprises both smooth surface 552 and textured surface 554 across different portions of a surface area thereof (here, a distal segment 529 comprises textured surface 554 while the remainder comprise smooth surface 552, though other arrangements are also contemplated). In examples including textured surfaces 554, the textured surface may increase friction between the probe and the target tissue or other ocular material by providing a higher coefficient of friction, thereby improving engagement of the probe with such tissue or material. Furthermore, with a higher coefficient of friction, the normal force needed to engage the tissue or material with the probe is reduced. And, as a result of reducing the normal force applied to the probe during a given procedure, the risk of injury or indentation to the eye may be reduced.

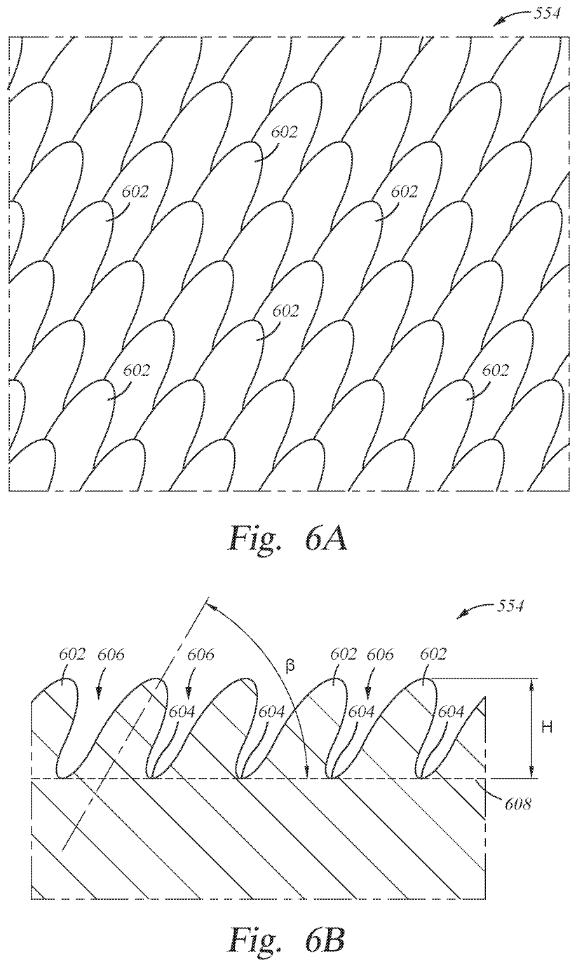

FIG. 6A illustrates an enlarged perspective view of textured surface 554 shown in FIGS. 5B, 5C, 5E, and 5F, in accordance with certain embodiments of the present disclosure. FIG. 6B illustrates a cross-sectional side view of textured surface 554, in accordance with certain embodiments of the present disclosure. For clarity, FIGS. 6A and 6B are herein described together.

As shown, textured surface 554 includes a plurality of raised surface features 602. Features 602 are configured to increase a coefficient of friction between a probe (e.g., probes 220a and 220b) and a target tissue or ocular material, thereby improving engagement between the probe and the target tissue or material during an ophthalmic procedure. In certain embodiments, features 602 comprise micro- or nano-posts. In certain embodiments, features 602 comprise micro- or nano-hooks. Features 602 may be arranged in any suitable arrangement on, e.g., an end face of a probe. For example, in certain embodiments, features 602 may be arranged in one or more linear arrays on an end face of a probe. In certain other embodiments, features 602 may be arranged in a circular or rotationally symmetric array on an end face of a probe. In the illustrated examples, features 602 may be formed by application of laser energy to the end face of a probe, e.g., probes 220a and 220b. In certain embodiments, a femtosecond or picosecond laser may be used.

Generally, features 602 have a height H that is measured from troughs 604 of traces 606 disposed between features 602, and that is further measured perpendicularly from effective surface 608 of textured surface 554, which is defined by a surface passing through troughs 604. In certain embodiments, the height H is between about 2 μm (micrometers) to about 10 μm, such as between about 3 μm and about 9 μm, such as between about 4 μm and about 8 μm, such as between about 5 μm and about 7 μm. In still other embodiments, the height H of features 602 may be greater than 10 μm or smaller than 2 μm. In further embodiments, the height H of features 602 may vary across textures surface 554.

In certain embodiments, features 602 are disposed at an angle θ incident to effective surface 608. Angling of features 602 may facilitate “grabbing” of the target tissue/material when the probe is moved against the target tissue/material in one direction, and “release” or the target tissue/material when the probe is moved against the target tissue/material in a second, opposite direction. Such bidirectional functionality makes probe engagement with target tissues/material efficient and predictable. In certain embodiments, the angle θ is within a range of 10° to 90°, where 90° is perpendicular to effective surface 608. In certain embodiments, the angle θ may be within a range of about 20° to about 70°, about 20° to about 55°, about 30° to about 60°, about 40° to about 50°, about 20° to about 50°, or about 30° to about 45°.

In summary, embodiments of the present disclosure generally relate to surgical probes for ophthalmic procedures. In particular, the embodiments herein provide probes designed for effective engagement and manipulation of the vitreous and other materials. Such probes include beveled probe tips that increase the area of vacuum generation at a target site without increasing probe gauge, thus facilitating improved tissue engagement as compared to other devices of similar gauge. In certain embodiments, the probes described herein further include one or more texturized surfaces for improved “grabbing” and manipulation of target tissues, and/or one or more surfaces formed of polymeric materials to reduce unwanted damage and enhance the safety thereof. Accordingly, the devices described herein address the deficiencies of certain existing methods and designs, and further reduce the risk of unwanted damage to peripheral tissues.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

1. A surgical probe for manipulating ocular tissues, comprising:
a handpiece configured to be held by a user; and
a tube defining a longitudinal axis, the tube comprising:
a proximal end coupled to the handpiece; and
a distal end opposite the proximal end and comprising a distal tip, wherein the distal tip is beveled and comprises an end face at least partially defining a port, the port having an elongated shape.

2. The surgical probe of claim 1, wherein the tube comprises a cylindrical morphology.

3. The surgical probe of claim 2, wherein the port has an ellipsoid shape.

4. The surgical probe of claim 1, wherein the tube comprises a curvature adjacent to the distal end that is configured to match a curvature of a retinal surface of an eye.

5. The surgical probe of claim 1, wherein the end face is substantially planar.

6. The surgical probe of claim 1, wherein the end face comprises an outward curvature configured to match a curvature of a retinal surface of an eye.

7. The surgical probe of claim 1, wherein the end face comprises a staggered profile having a plurality of stepped segments.

8. The surgical probe of claim 1, wherein the end face is disposed at an angle between about 10° and about 30° relative to a normal of the longitudinal axis.

9. The surgical probe of claim 8, wherein the face is disposed at an angle of 18° relative to a normal of the longitudinal axis.

10. The surgical probe of claim 1, wherein the tube is formed of a plastic or polymeric material.

11. The surgical probe of claim 1, wherein the tube is formed of aluminum, stainless steel, Phynox, Elgiloy, nitinol, or other metal alloy.

12. The surgical probe of claim 1, wherein the tube comprises a distal portion formed of a plastic or polymeric material and a proximal portion formed of aluminum, stainless steel, Phynox, Elgiloy, nitinol, or other metal alloy.

13. The surgical probe of claim 1, wherein at least a portion of the end face comprises a textured surface having a plurality of raised surface features.

14. The surgical probe of claim 13, wherein the raised surface features comprise nano-hooks.

15. The surgical probe of claim 13, wherein the raised surface features are angled relative to an effective surface of the textured surface to facilitate grabbing of an ocular tissue when the probe is moved in a first direction and release of the ocular tissue when the probe is moved in a second direction.
